(19) United States
(12) Patent Application Publication
(10) Pub. No.: US 2026/0279590 A1
(43) Pub. Date: Sep. 17, 2026

(54) HEALTH CONDITION ESTIMATION SYSTEM, INFORMATION PROCESSING APPARATUS, HEALTH CONDITION ESTIMATION METHOD, AND NON-TRANSITORY COMPUTER READABLE MEDIUM

(71) Applicant: Yokogawa Electric Corporation, Tokyo (JP)

(72) Inventors: Kiyomi Muraoka, Tokyo (JP); Hideo Uemura, Tokyo (JP); Kie Shimamura, Tokyo (JP)

(73) Assignee: Yokogawa Electric Corporation, Tokyo (JP)

(21) Appl. No.: 19/557,274

(22) Filed: Mar. 5, 2026

(30) Foreign Application Priority Data
Mar. 12, 2025 (JP) 2025-039780

(51) Int. Cl. G16H 50/30; A61B 5/00; A61B 5/16; G16H 50/50

(52) U.S. Cl. CPC G16H 50/30; A61B 5/16; A61B 5/4064; G16H 50/50

(57) ABSTRACT

A health condition estimation system includes a brain wave sensor configured to detect brain waves of a user and output brain wave information, a behavior sensor configured to collect and output behavior information for the user, and an information processing apparatus. The information processing apparatus includes a processor that estimates the health condition of the user based on the brain wave information and the behavior information and outputs information on the health condition of the user.

### CROSS REFERENCE TO RELATED APPLICATION

The present application claims priority to Japanese Patent Application No. 2025-39780 filed on Mar. 12, 2025, the entire contents of which are incorporated herein by reference.

### TECHNICAL FIELD

The present disclosure relates to a health condition estimation system, an information processing apparatus, a health condition estimation method, and a non-transitory computer readable medium.

### BACKGROUND

As described in Patent Literature (PTL) 1, a known apparatus detects whether a target of a user is different from normal, based on the results of measuring biological information for the user or the environmental information around the user, and the results of a medical interview with the user.

### Citation List

### Patent Literature

PTL 1: JP 2008-011865 A

### SUMMARY

A health condition estimation system according to an embodiment of the present disclosure includes a brain wave sensor configured to detect brain waves of a user and output brain wave information, a behavior sensor configured to collect and output behavior information for the user, and an information processing apparatus including a processor. The processor is configured to estimate a health condition of the user, based on the brain wave information and the behavior information, and output information on the health condition of the user.

An information processing apparatus according to an embodiment of the present disclosure includes a processor configured to estimate a health condition of a user. The processor is configured to acquire brain wave information from a brain wave sensor that detects brain waves of the user and outputs the brain wave information, and acquire behavior information for the user. The processor is configured to estimate a health condition of the user, based on the brain wave information and the behavior information, and output information on the health condition of the user.

A health condition estimation method according to an embodiment of the present disclosure is a method to be executed by a processor and includes acquiring brain wave information from a brain wave sensor configured to detect brain waves of a user, estimating a health condition of the user based on the brain wave information and the behavior information, and outputting information on the health condition of the user.

A non-transitory computer readable medium according to an embodiment of the present disclosure stores a program configured to cause a processor to execute the aforementioned health condition estimation method.

### BRIEF DESCRIPTION OF THE DRAWINGS

In the accompanying drawings:

FIG. 1 is a diagram illustrating a schematic configuration of a health condition estimation system according to an embodiment;

FIG. 2 is a diagram illustrating how a health condition is estimated using an estimation model;

FIG. 3 is a flowchart illustrating an example of the operation of the health condition estimation system according to an embodiment;

FIG. 4 is a diagram illustrating a schematic configuration of a health condition estimation system according to another embodiment;

FIG. 5 is a diagram illustrating an example of a configuration for correcting the output of an estimation model using the output of an attribute model; and

FIG. 6 is a diagram illustrating an example of a configuration for correcting parameters of an estimation model using the output of an attribute model.

### DETAILED DESCRIPTION

When a user whose health condition is to be managed is interviewed, the results of interviewing the user are the user's subjective opinion. It is difficult to continuously ask questions of users, because doing so places a burden on the users. It is also difficult to increase the frequency of health checkups for users, as this too places a burden on the users. Demand exists for objectively and continuously estimating a user's health condition. According to the health condition estimation system, information processing apparatus, health condition estimation method, and program of the present disclosure, a health condition of a user is estimated objectively and continuously.

(1) A health condition estimation system according to an embodiment of the present disclosure includes a brain wave sensor configured to detect brain waves of a user and output brain wave information, a behavior sensor configured to collect and output behavior information for the user, and an information processing apparatus including a processor. The processor is configured to estimate a health condition of the user, based on the brain wave information and the behavior information, and output information on the health condition of the user. With this configuration, the user's daily health condition can be estimated without increasing the frequency of the user's medical interviews or health checkups. As a result, it becomes possible to objectively and continuously estimate the health condition of the user.

(2) In the health condition estimation system according to (1), the information processing apparatus may further include a memory configured to store an estimation model created by machine learning based on training data. The estimation model may be a model configured to output the information on the health condition when the brain wave information and the behavior information are inputted. The processor may be configured to output the information on the health condition by inputting the brain wave information and the behavior information into the estimation model. This configuration improves the accuracy of estimating the health condition of the user.

(3) In the health condition estimation system according to (2), the information processing apparatus may include an input interface configured to acquire attribute information related to attributes of the user. The memory may be configured to store an attribute model created by machine learning based on attribute training data. The attribute model may be a model configured to output correction information for correcting the information on the health condition when the attribute information is inputted. The processor may be configured to correct the information on the health condition using the correction information. This configuration improves the accuracy of estimating the health condition of the user.

(4) In the health condition estimation system according to (3), the processor may be configured to correct an output of the estimation model using the correction information and to output corrected information as the information on the health condition. This configuration improves the accuracy of estimating the health condition of the user.

(5) In the health condition estimation system according to (3), in a case in which the estimation model includes processing layers, the processor may be configured to correct parameters of at least one processing layer among the processing layers using the correction information. This configuration improves the accuracy of estimating the health condition of the user.

(6) In the health condition estimation system according to any one of (1) to (5), the information on the health condition may include a stress level of the user. With this configuration, users can understand their own stress.

(7) In the health condition estimation system according to any one of (1) to (6), the information processing apparatus may further include an output interface. The processor may be configured to generate a behavior proposal to propose to the user based on the information on the health condition and display the generated behavior proposal on the output interface. With this configuration, users can adopt appropriate behaviors according to their health condition.

(8) In the health condition estimation system according to (7), the processor may be configured to acquire a schedule of the user and generate the behavior proposal based on the acquired schedule and the information on the health condition. With this configuration, an appropriate behavior proposal is generated according to the user's schedule.

(9) An information processing apparatus according to an embodiment of the present disclosure includes a processor configured to estimate a health condition of a user. The processor is configured to acquire brain wave information from a brain wave sensor that detects brain waves of the user and outputs the brain wave information, and acquire behavior information for the user. The processor is configured to estimate a health condition of the user, based on the brain wave information and the behavior information, and output information on the health condition of the user.

(10) A health condition estimation method according to an embodiment of the present disclosure is a method to be executed by a processor and includes acquiring brain wave information from a brain wave sensor configured to detect brain waves of a user, acquiring behavior information for the user, estimating a health condition of the user based on the brain wave information and the behavior information, and outputting information on the health condition of the user.

(11) A non-transitory computer readable medium according to an embodiment of the present disclosure stores a program configured to cause a processor to execute the health condition estimation method according to (10).

In order to manage the health condition of a subject, a health examination based on testing may be performed at a medical institution periodically, for example, once a year. In addition, the subject may be asked to undergo a medical interview, such as a stress check. The results of the health examination and the medical interview are checked by a doctor, such as an industrial physician, and may be used in guidance or consultation regarding the health of the subject.

However, when managing the health condition of a subject using the above-described method, the following problems may arise.

Even if the health condition at the time of testing is determined, the health condition between tests is not determined.

The results of interviewing the subject are a subjective evaluation by the subject. Therefore, there is a risk that any disorders that subjects themselves are not aware of may be overlooked. Furthermore, there is a risk that subjects may be concerned about others seeing the results of their medical interviews and thus might not be able to perform a correct self-evaluation.

In order to understand changes in physical condition or implement health management such as recommended behaviors, specialized knowledge and guidance from a doctor are required. Therefore, it is difficult for subjects to notice disorders themselves.

The subject only recognizes the need for action after some sort of disorder manifests.

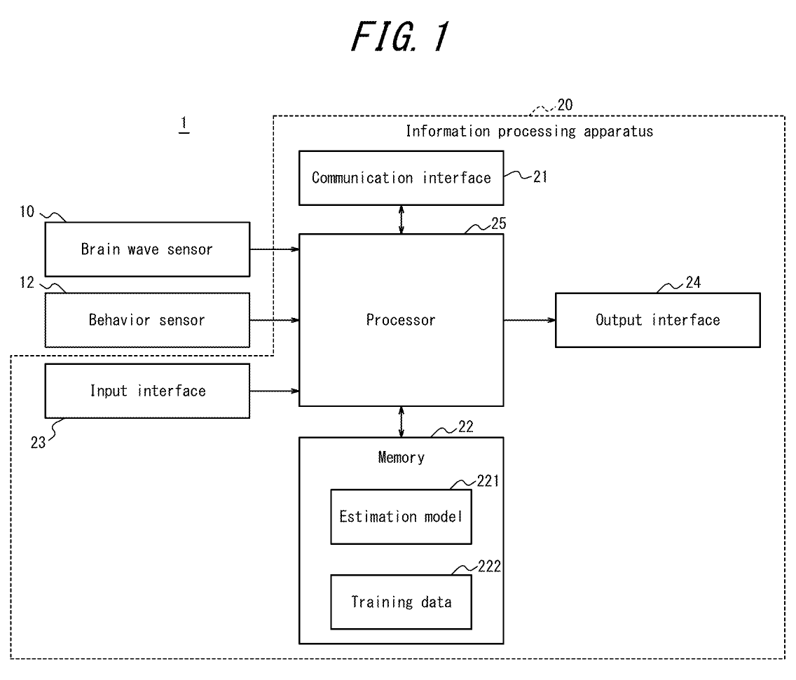

In contrast, the health condition estimation system 1 and information processing apparatus 20 (see FIG. 1), along with the health condition estimation method and program, according to the present disclosure objectively and continuously estimate the health condition of a user, such as a subject whose health condition is to be managed. An embodiment of the present disclosure will be described below with reference to the drawings.

### Configuration example of health condition estimation system 1

As illustrated in FIG. 1, a health condition estimation system 1 according to an embodiment of the present disclosure includes a brain wave sensor 10, a behavior sensor 12, and an information processing apparatus 20. The brain wave sensor 10 and the behavior sensor 12 can communicate with the information processing apparatus 20 via wireless communication or wired communication.

The health condition estimation system 1 can estimate the health condition of a user. The user may, for example, be a desk worker who operates the information processing apparatus 20 for work.

The brain wave sensor 10 is a sensor that detects the brain waves of a user whose health condition is to be estimated and outputs brain wave information. Upon detecting brain waves of the user, the brain wave sensor 10 transmits brain wave information to the information processing apparatus 20.

The brain wave sensor 10 is attached to a user whose health condition is to be estimated and thereby detects the brain waves of the user, outputting the result of detecting brain waves as brain wave information. The brain wave sensor 10 may be any brain wave sensor capable of detecting the brain waves of the user. The brain wave sensor 10 may be an earphone-type brain wave sensor, but this example is not limiting.

The brain wave information includes the detection results of the waveforms of weak electrical signals generated from the brain. The brain wave information includes information on the frequency bands in which brain waves are appearing more prominently, or information on the reaction time to stimuli to the sensory organs such as hearing or vision, and is useful for estimating the brain's activity state, the user's state of concentration or fatigue, or the like.

The behavior sensor 12 collects behavior information for the user. The behavior information for the user may include information about the user's actual behavior, such as the amount of sleep or the number of steps taken. The behavior information for the user may include the activity schedule or the like of the user. The behavior sensor 12 may be a wearable device that is attached to the user's body.

The behavior information for the user is used to estimate the health condition of the user. For example, if the user does not get enough sleep, the user's level of fatigue may increase. Furthermore, lack of exercise or excessive exercise may have a negative impact on the health condition of the user.

The health condition estimation system 1 may include sensors that measure anthropometric data of the user. Anthropometric data includes, but is not limited to, heart rate information, body temperature, and blood oxygen level.

Heart rate information is information relating to the beating of the heart. Heart rate information may be used to calculate indices such as heart rate or heart rate variability. These indices are useful for estimating health conditions, such as balance in the autonomic nervous system, or stress levels. Body temperature is useful as an indicator of fever or poor health. The blood oxygen concentration reflects the functional state of the respiratory system and is useful for estimating fatigue or stress levels during exercise.

The health condition estimation system 1 may include a sensor that collects environmental information around the user. The environmental information may include temperature, humidity, air pressure, or the like. The environmental information may include the illuminance of indoor lighting, the illuminance of outdoor sunlight, and the like. The environmental information may include the loudness of noise. The environmental information is not limited to these examples and may include various other information. The environmental information may be used to estimate the health condition of the user. For example, noise increases stress for users. Temperature, humidity, and air pressure can affect the physical condition of the user. Illuminance can be a factor in causing stress or fatigue to the user.

The information processing apparatus 20 may be a device used by the user whose health condition is to be estimated. For example, if the user is a desk worker, the information processing apparatus 20 may be a device used by the user for work. The information processing apparatus 20 can estimate the health condition of the user using the brain wave information acquired from the brain wave sensor 10 and the behavior information acquired from the behavior sensor 12.

The information processing apparatus 20 may be a mobile terminal such as a smartphone carried by a user. The information processing apparatus 20 may be a wearable device such as a smartwatch that is worn on the user's body. The information processing apparatus 20 may be a general-purpose electronic device such as a personal computer (PC) or a tablet terminal.

The information processing apparatus 20 includes a communication interface 21, a memory 22, an input interface 23, an output interface 24, and a processor 25.

The communication interface 21 includes a communication module. For example, the communication interface 21 may include a communication module compatible with a LAN (Local Area Network). In one embodiment, the information processing apparatus 20 is connected to a network via the communication interface 21. The communication interface 21 may be capable of communicating with a server or the like via the network.

The memory 22 is, for example, a semiconductor memory, a magnetic memory, an optical memory, or the like, but is not limited to these. The memory 22 may function as, for example, a main memory device, an auxiliary memory device, or a cache memory. The memory 22 stores any information used in the operation of the information processing apparatus 20. For example, the memory 22 may store system programs, application programs, various types of information, and the like. A portion of the memory 22 may be installed outside the information processing apparatus 20. In this case, a portion of the externally installed memory 22 may be connected to the information processing apparatus 20 via any appropriate interface.

The input interface 23 may include one or more interfaces for input that receive an operation from the user and acquire input information based on the user operation. For example, the input interface 23 may include at least one of a keyboard, a mouse, or a touch screen that is integrated with the display of the output interface 24, but is not limited to these.

The output interface 24 includes one or more interfaces for output that output information to notify the user. For example, the output interface 24 includes a display that outputs information as an image, a speaker that outputs information as sound, or the like, but is not limited to these.

The processor 25 is a general-purpose processor, such as a Central Processing Unit (CPU) or a Graphics Processing Unit (GPU), or a dedicated processor that is dedicated to specific processing. The processor 25 may be a Field-Programmable Gate Array (FPGA), a Digital Signal Processor (DSP), an Application Specific Integrated Circuit (ASIC), or the like. The processor 25 may be a combination of a plurality of processors. The processor 25 executes processes related to the operation of the information processing apparatus 20 while controlling each component of the information processing apparatus 20.

The processor 25 includes an interface for acquiring data from the brain wave sensor 10 and the behavior sensor 12. The interface may be configured to be capable of communicating with other devices based on a wireless communication standard such as Bluetooth^{®} (Bluetooth is a registered trademark in Japan, other countries, or both). The interface may be configured to be capable of communicating with other devices based on a serial communication standard such as RS-232C or RS-485. The interface is not limited to these examples and may be configured to be capable of communicating with other devices based on various communication standards.

The interface is not limited to the brain wave sensor 10 and the behavior sensor 12 and may be configured to be capable of communicating with sensors that measure anthropometric data of the user.

The functions of the information processing apparatus 20 can be realized by causing the processor 25 to execute a program stored in the memory 22. That is, the functions of the information processing apparatus 20 can be realized by software.

### Example of operation of health condition estimation system 1

An example of the operation of the health condition estimation system 1 according to the present disclosure is described below.

The brain wave sensor 10 detects the brain waves of a user whose health condition is to be estimated. Upon detecting brain waves of the user, the brain wave sensor 10 transmits brain wave information to the information processing apparatus 20.

The behavior sensor 12 collects behavior information for the user and transmits the collected behavior information to the information processing apparatus 20.

The processor 25 of the information processing apparatus 20 acquires the brain wave information transmitted by the brain wave sensor 10 and the behavior information transmitted by the behavior sensor 12. When the brain wave sensor 10 or the behavior sensor 12 is communicating with the communication interface 21, the processor 25 may acquire the brain wave information transmitted by the brain wave sensor 10, or the behavior information transmitted by the behavior sensor 12, via the communication interface 21.

As illustrated in FIG. 1, the memory 22 stores an estimation model 221 and training data 222.

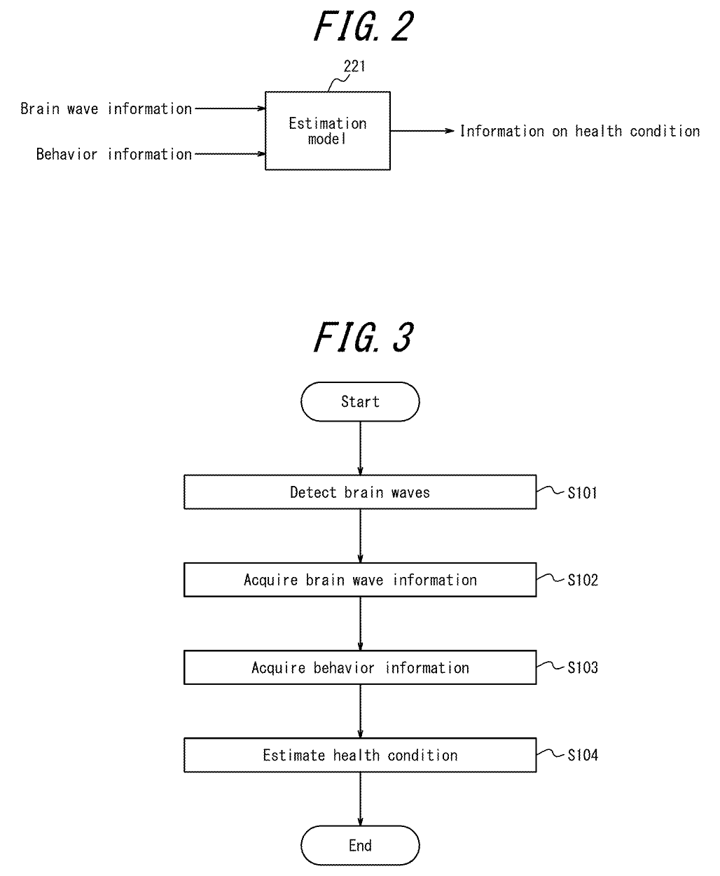

The estimation model 221 is a model for estimating the health condition of the user based on the brain wave information acquired from the brain wave sensor 10 and the behavior information acquired from the behavior sensor 12. As illustrated in FIG. 2, when the brain wave information and the behavior information are inputted, the estimation model 221 estimates the health condition of the user and outputs information on the health condition of the user.

The estimation model 221 may be configured to allow input of anthropometric data of the user or information on the surrounding environment of the user. The estimation model 221 may estimate the health condition of the user and output the information on the health condition of the user when anthropometric data or environmental information is inputted in addition to the brain wave information and the behavior information. This configuration improves the accuracy of estimating the health condition of the user.

The estimation model 221 may be a model trained by machine learning based on the training data 222. The training data 222 is a data set used by the estimation model 221 when performing machine learning. The training data 222 may be a data set that associates past brain wave information and behavior information for the user with past information on the health condition of the user. In a case in which the estimation model 221 is a model created by machine learning, the accuracy of estimating the health condition improves as the amount of accumulated past data increases. Past anthropometric data or past environmental information for the user may be further associated in the training data 222. This configuration improves the accuracy of estimating the health condition of the user.

The estimation model 221 is not limited to a model created by machine learning. The estimation model 221 may, for example, be a regression model.

In the example illustrated in FIG. 1, the memory 22 stores the training data 222, but this is just an example. The training data 222 may be stored in an apparatus other than the information processing apparatus 20, for example.

Upon acquiring the brain wave information from the brain wave sensor 10 and the behavior information from the behavior sensor 12, the processor 25 estimates the health condition of the user in real time based on the brain wave information and the behavior information and outputs information on the health condition of the user. The processor 25 inputs the brain wave information and behavior information into the estimation model 221 stored in the memory 22, thereby estimating the health condition of the user and outputting information on the estimated health condition of the user.

The information on the health condition of the user includes various types of information relating to the health condition. The information on the health condition may include, for example, the stress level of the user. The stress level of the user may, for example, be a numerical representation of the stress on the user. The information on the health condition may include, for example, the fatigue level of the user. The fatigue level of the user may be, for example, a numerical representation of how physically or mentally tired the user is. The information on the health condition may include, for example, the level of sleepiness of the user. The level of sleepiness of the user may be a numerical representation of how sleepy the user feels. The level of sleepiness of the user may be a numerical value representing not only the sleepiness that the user is aware of, but also the sleepiness that is potentially occurring but that the user is not aware of.

Upon estimating the health condition of the user, the processor 25 may cause the output interface 24 to display information on the estimated health condition of the user. This enables users to understand information on their own health condition in real time.

The processor 25 may also generate a behavior proposal to propose to the user based on the information on the health condition outputted by the estimation model 221. For example, if the information on the health condition indicates that the user has a high level of stress, fatigue, or sleepiness, a behavior proposal such as taking a break, stretching, exercising, or finishing work early may be generated.

Upon generating the behavior proposal, the processor 25 may cause the output interface 24 to display the behavior proposal. This enables users to adopt appropriate behaviors according to their own health condition and reduce their stress level.

When displaying the behavior proposal on the output interface 24, the processor 25 may display the behavior proposal together with the information on the health condition. This enables users to check their own health condition and appropriate behaviors at the same time.

When generating the behavior proposal, the processor 25 may acquire the schedule of the user and generate the behavior proposal based on the acquired schedule and the information on the health condition outputted by the estimation model 221. In this case, the processor 25 may acquire the schedule of the user from a terminal device such as a smartphone used by the user, or may acquire the schedule of the user from a server that stores the schedule of the user.

For example, when the schedule of the user includes a meeting, the processor 25 may generate a behavior proposal to reduce the stress level or degree of fatigue before the start of the meeting, such as, "You have a meeting at 4 p.m. Why not take a break and refresh yourself before the meeting?"

This enables the processor 25 to generate appropriate behavior proposals according to the schedule of the user.

The health condition estimation system 1 may execute a health condition estimation method including the steps of the flowchart illustrated in FIG. 3. The health condition estimation method may be realized as a program to be executed by the processor 25 of the information processing apparatus 20. The program may be stored in a non-transitory computer readable medium such as an electromagnetic storage medium.

Step S101: the brain wave sensor 10 detects brain waves of the user who is operating the information processing apparatus 20. Upon detecting brain waves of the user, the brain wave sensor 10 transmits brain wave information to the information processing apparatus 20.

Step S102: the processor 25 acquires the brain wave information from the brain wave sensor 10.

Step S103: the processor 25 acquires the behavior information from the behavior sensor 12.

Step S104: the processor 25 estimates the health condition of the user based on the brain wave information and the behavior information. The processor 25 may cause the output interface 24 to display the information on the estimated health condition of the user.

According to the health condition estimation system 1 of the embodiment described above, the accuracy of estimating the health condition of the user can be improved. Specifically, the health condition estimation system 1 includes the brain wave sensor 10 that detects the brain waves of the user and outputs brain wave information, the behavior sensor 12 that collects and outputs behavior information for the user, and the information processing apparatus 20. The information processing apparatus 20 includes the processor 25 that estimates the health condition of the user based on the brain wave information and the behavior information and outputs information on the health condition of the user. In this way, the information processing apparatus 20 estimates the health condition of the user by taking into consideration not only the brain wave information, which is the biological information for the user, but also behavior information for the user. Therefore, according to the health condition estimation system 1 of the present embodiment, it is possible to improve the estimation accuracy in estimating a health condition based on brain wave information.

The health condition estimation system 1 according to the present embodiment can also estimate the user's daily health condition without increasing the frequency of the user's medical interviews or health checkups. As a result, it becomes possible to objectively and continuously estimate the health condition of the user.

The health condition estimation system 1 according to the present embodiment estimates the health condition of the user based on the brain wave information and the behavior information and therefore can detect stress, fatigue, or drowsiness in the user at an early stage.

The health condition estimation system 1 according to the present embodiment may generate a behavior proposal to propose to the user based on the information on the health condition and cause the generated behavior proposal to be displayed on the output interface 24. If the user implements the suggested behavior in response, the user's stress or fatigue may be reduced and the user's concentration and productivity may be improved.

### Other embodiment

In another embodiment, the health condition estimation system 1 may estimate the health condition of a user by taking into account the attributes of the user. The attributes are information that represent the characteristics of the user. The attributes are used, for example, to classify users having the same attributes into a group. In the present embodiment, the attributes may include information that can reveal some tendency or commonality in the correlation between brain wave information and information on the health condition for users with the same attribute.

The attributes may include, for example, the age, sex, or physical characteristics of the user. Age may be expressed as the numerical value of age itself, or may be expressed by classification into age groups such as 40s or categories such as young, middle-aged, or elderly. The physical characteristics include the height, weight, obesity level, and the like of the user and may be expressed by classifying the numerical value of the height, weight, obesity level, and the like into a plurality of categories. The physical characteristics may be expressed as a combination of numerical values such as height, weight, or obesity level.

The attributes may include, for example, the work style of the user. The work style may be classified by whether the employee works in the office, at home, or the like. The work style may be classified as fixed working hours, flextime, reduced working hours, or the like.

The attributes may include, for example, the occupation of the user. The occupation may be classified as desk work, manual labor, or the like.

The attributes may include, for example, the job position of the user. The job position may be expressed by classification into superior or subordinate, or may be expressed by classification into a hierarchy such as a section chief or department chief.

The attributes are not limited to the examples given above and may be expressed by various other characteristics.

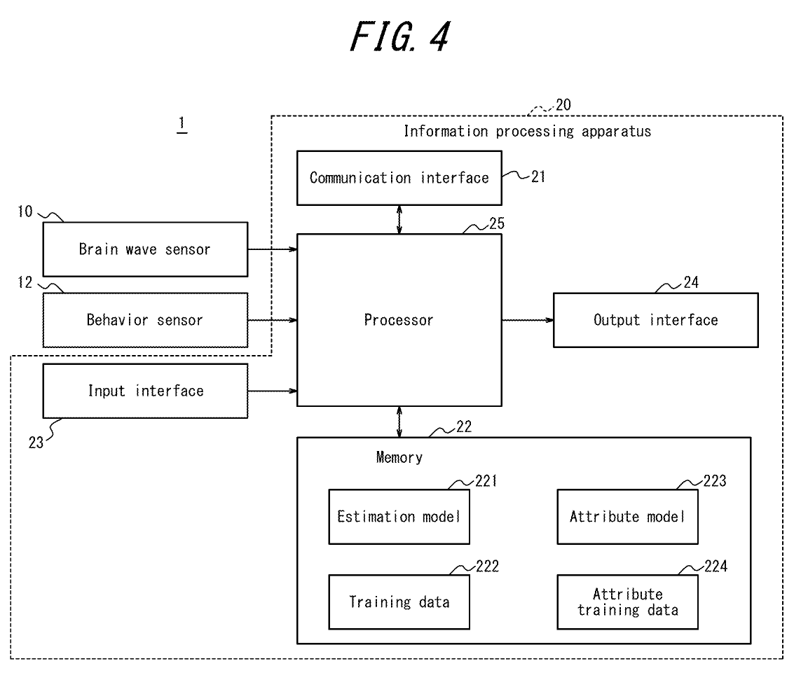

As illustrated in FIG. 4, in the health condition estimation system 1 according to the present embodiment, the memory 22 of the information processing apparatus 20 stores an attribute model 223 and attribute training data 224. When an attribute of the user is inputted, the attribute model 223 outputs information regarding the tendency or commonality of the correlation between the brain wave information and the information on the health condition found among a plurality of users belonging to a group classified by that attribute.

The attribute model 223 may be a model created by machine learning based on the attribute training data 224. The attribute training data 224 is a data set used by the attribute model 223 when performing machine learning. The attribute training data 224 may be a data set that associates the attributes of the user with past brain wave information and information on the health condition for users who belong to the group classified by those attributes. In a case in which the attribute model 223 is a model created by machine learning, the accuracy of estimating the health condition improves as the amount of accumulated past data increases.

The attribute model 223 is not limited to a model created by machine learning. The attribute model 223 may, for example, be a regression model.

In the example illustrated in FIG. 4, the memory 22 stores the attribute training data 224, but this is just an example. The attribute training data 224 may be stored in an apparatus other than the information processing apparatus 20, for example.

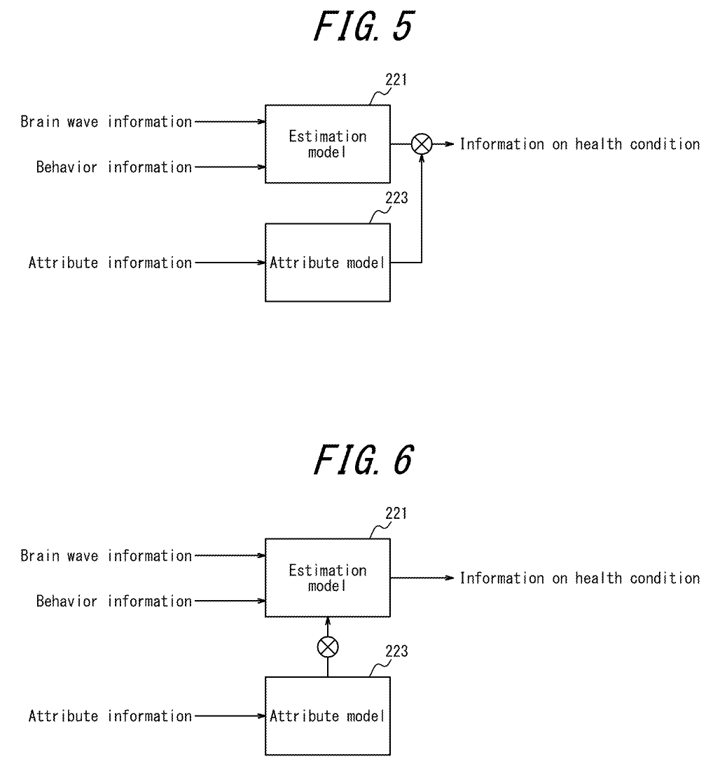

The information outputted from the attribute model 223 may be used to correct the output of the estimation model 221, for example, as illustrated in FIG. 5. The processor 25 may perform a calculation between the output of the estimation model 221 and the output of the attribute model 223 and output the result as information on the health condition. When the output of the estimation model 221 is expressed in vector format, the information outputted from the attribute model 223 may be in matrix format.

The information outputted from the attribute model 223 may be inputted to the estimation model 221 and used to correct the parameters inside the estimation model 221, as illustrated in FIG. 6, for example. In a case in which the estimation model 221 is a neural network model that includes a plurality of processing layers therein, the processor 25 may use information outputted from the attribute model 223 to correct the weighting parameters of at least one processing layer within the estimation model 221. The information outputted from the attribute model 223 may be coefficients by which the weighting parameters of the processing layer in the estimation model 221 are respectively multiplied, or the information may be a common coefficient by which each of the weighting parameters is multiplied.

In a case in which the estimation model 221 is a regression model, the processor 25 may use information outputted from the attribute model 223 to correct the coefficients included in the mathematical formula of the regression model. The information outputted from the attribute model 223 may be coefficients by which the coefficients included in the mathematical formula of the regression model are respectively multiplied, or the information may be a common coefficient by which each of the coefficients is multiplied.

The information outputted from the attribute model 223 is not limited to the above-described examples and may be information in various forms. The information outputted from the attribute model 223 is used to correct the information on the health condition, both in the case of using the information to correct the output of the estimation model 221 and the case of using the information to correct the parameters inside the estimation model 221. Therefore, the information outputted from the attribute model 223 is also referred to as corrected information on the health condition.

The processor 25 may receive attribute information to be inputted to the attribute model 223 through input from the user via the input interface 23. The processor 25 may acquire the attribute information for the user by presenting the user with questions regarding attributes of the user and accepting the answers from the user during user registration. When the attributes of the user change, the processor 25 may acquire the change in the attribute information for the user by the user self-reporting. The processor 25 may periodically request that the user input the attribute information. The processor 25 may acquire user attribute information from another apparatus.

The attribute model 223 may be configured to receive input of brain wave information in addition to attribute information.

The attribute training data 224 may be a data set that is further associated with past behavior information for the user. In this case, the attribute model 223 may be configured to receive input of behavior information in addition to the attribute information and brain wave information.

The processor 25 may generate a behavior proposal taking into account the attribute information of the user.

As described above, the health condition estimation system 1 can improve the accuracy of estimating the health condition of a user by taking into account attribute information of the user.

It will be apparent to those skilled in the art that the present disclosure may be realized in certain forms other than the above embodiments without departing from the spirit or essential characteristics of the present disclosure. Accordingly, the foregoing description is merely illustrative and is not limiting. The scope of the disclosure is defined by the appended claims, not by the foregoing description. Among all modifications, those within a range of equivalents to the present disclosure shall be considered as being included in the present disclosure.

For example, the arrangement and number of each of the above-mentioned components are not limited to those illustrated in the above description and the drawings. The arrangement and number of each component may be configured freely as long as the corresponding function can be realized.

For example, a general-purpose electronic device such as a smartphone or a computer may be configured to function as the information processing apparatus 20 according to the above embodiment. Specifically, a program describing the processing details to realize each function of the information processing apparatus 20 according to the embodiment could be stored in the memory of the electronic device, and the program could be read and executed by the processor of the electronic device. An embodiment of the present disclosure can thus also be realized as a program executable by a processor.

For example, part of the processing executed by the information processing apparatus 20 in the above embodiment may be executed on a server capable of communicating with the information processing apparatus 20. For example, the information processing apparatus 20 may transmit the brain wave information and the behavior information to the server, and the server may execute processing to estimate the health condition of the user based on the brain wave information and the behavior information acquired from the information processing apparatus 20.

For example, in the above embodiment, the information processing apparatus 20 acquires brain wave information, but the information processing apparatus 20 may acquire biological information other than brain wave information together with the brain wave information. The biological information other than brain waves may, for example, be heart rate, blood pressure, or body temperature.

1. A health condition estimation system comprising:
an information processing apparatus comprising a brain wave sensor configured to detect brain waves of a user and output brain wave information, a behavior sensor configured to collect and output behavior information for the user, and a processor, wherein
the processor is configured to estimate a health condition of the user, based on the brain wave information and the behavior information, and output information on the health condition of the user.

2. The health condition estimation system according to claim 1, wherein
the information processing apparatus further comprises a memory configured to store an estimation model created by machine learning based on training data,
the estimation model is a model configured to output the information on the health condition when the brain wave information and the behavior information are inputted, and
the processor is configured to output the information on the health condition by inputting the brain wave information and the behavior information into the estimation model.

3. The health condition estimation system according to claim 2, wherein
the information processing apparatus comprises an input interface configured to acquire attribute information related to attributes of the user,
the memory is configured to store an attribute model created by machine learning based on attribute training data,
the attribute model is a model configured to output correction information for correcting the information on the health condition when the attribute information is inputted, and
the processor is configured to correct the information on the health condition using the correction information.

4. The health condition estimation system according to claim 3, wherein the processor is configured to correct an output of the estimation model using the correction information and to output corrected information as the information on the health condition.

5. The health condition estimation system according to claim 3, wherein in a case in which the estimation model includes processing layers, the processor is configured to correct parameters of at least one processing layer among the processing layers using the correction information.

6. The health condition estimation system according to claim 1, wherein the information on the health condition includes a stress level of the user.

7. The health condition estimation system according to claim 1, wherein
the information processing apparatus further comprises an output interface, and
the processor is configured to generate a behavior proposal to propose to the user based on the information on the health condition and display the generated behavior proposal on the output interface.

8. The health condition estimation system according to claim 7, wherein the processor is configured to acquire a schedule of the user and generate the behavior proposal based on the acquired schedule and the information on the health condition.

9. An information processing apparatus comprising:
a processor configured to
acquire brain wave information from a brain wave sensor that detects brain waves of the user and outputs the brain wave information, and acquire behavior information for the user, and
estimate a health condition of the user, based on the brain wave information and the behavior information, and output information on the health condition of the user.

10. A health condition estimation method to be executed by a processor, the method comprising:
acquiring brain wave information from a brain wave sensor configured to detect brain waves of a user;
acquiring behavior information for the user; and
estimating a health condition of the user, based on the brain wave information and the behavior information, and outputting information on the health condition of the user.

11. A non-transitory computer readable medium storing a program configured to cause a processor to execute the health condition estimation method according to claim 10.
